(19) United States
(12) Patent Application Publication
(10) Pub. No.: US 2002/0164657 A1
(43) Pub. Date: Nov. 7, 2002

(54) Assays for assessing A beta-Tau aggregation

(76) Inventors: Satish K. Sharma, Portage, MI (US); Kenneth B. Rank, Mattawan, MI (US)

Correspondence Address: PHARMACIA & UPJOHN, 301 HENRIETTA ST, 0228-32-LAW, KALAMAZOO, MI, 49007, US

(21) Appl. No.: 10/076,708

(22) Filed: Feb. 15, 2002

Related U.S. Application Data
(60) Provisional application No. 60/271,102, filed on Feb. 23, 2001.

(51) Int. Cl. G01N 33/53; G01N 33/567; G01N 33/537; G01N 33/543

(52) U.S. Cl. 435/7.2

(57) ABSTRACT

The present invention deals with the field of cell biology, more specifically the field of neuronal cell biology and neurodegenerative diseases. The invention provides for assays assessing Aβ-Tau aggregation

### CROSS REFERENCE TO RELATED APPLICATIONS

This application claims the benefit of the following provisional application: Application Serial No. 60/271102 filed Feb. 23, 2001 under 35 U.S.C 119(e)(1).

### FIELD OF THE INVENTION

The present invention deals with the field of cell biology, more specifically the field of neuronal cell biology and neurodegenerative diseases. The invention provides for assays assessing Aβ-Tau aggregation

### BACKGROUND

In Alzheimer's disease, Aβ peptide and hyperphosphorylated tau are found in the amyloid plaques and neurofibrillary tangles, respectively. Recently, it has been suggested that Aβ peptide oligomerization begins intraneuronally and the earliest detectable dementia in AD arises from the onset of tau aggregation. Six isoforms of human tau are expressed in adult human brain. (1) These arise from alternate splicing of the mRNA transcribed from a single gene located on the long arm of chromosome 17 (1-5) One of the functions of the tau protein is to promote microtubule assembly in vivo and to stabilize microtubules in the nervous system (6-10).

Neurofibrillary tangles (NFT) and senile plaques constitute two prominent neuropathological characteristics of Alzheimer's disease (11,12). The main fibrous component of all neurofibrillary lesions is paired helical filament (PHF) which contains predominantly the abnormally phosphorylated tau (13-18). It has been hypothesized that aberrant phosphorylation of tau leads to its aggregation into PHF, resulting in destabilization of microtubules and the death of neurons (1,19). Notably, hyperphosphorylated tau has been detected in other tau-positive filamentous lesions, in a group of diseases collectively known as the tauopathies. These neurodegenerative diseases include Progressive Supranuclear Palsy (PSP), Corticobasal Degeneration (CBD), Down's syndrome, Frontotemporal dementia and Parkinsonism linked to chromosome 17 (FTDP), and Pick's disease (20). Recent studies show that these various phenotypes might be the result of phosphorylation of specific tau isoforms in different nerve cells in distinct brain regions (21).

The upstream factors and small molecule modulators of tau aggregation have remained elusive however. Assays to access this phenomenon are a valuable tool in the discovery of agents potentially useful in the treatment and prevention of a variety of neurodegenerative diseases.

### References Cited

1. Goedert, M., et al., Multiple isoforms of human microtubule-associated protein tau: sequences and localization in neurofibrillary tangles of Alzheimer's disease. Neuron, 1989. 3(4): p. 519-26.

2. Lee, G., N. Cowan, and M. Kirschner, The primary structure and heterogeneity of tau protein from mouse brain. Science, 1988. 239(4837): p. 285-8.

3. Goedert, M., et al., Cloning and sequencing of the cDNA encoding a core protein of the paired helical filament of Alzheimer disease: identification as the microtubule-associated protein tau. Proc Natl Acad Sci U.S.A., 1988. 85(11): p. 4051-5.

4. Himmler, A., Structure of the bovine tau gene: alternatively spliced transcripts generate a protein family. Mol Cell Biol, 1989. 9(4): p. 1389-96.

5. Kosik, K. S., C. L. Joachim, and D. J. Selkoe, Microtubule-associated protein tau (tau) is a major antigenic component of paired helical filaments in Alzheimer disease. Proc Natl Acad Sci U.S.A., 1986. 83(11): p. 4044-8.

6. Caceres, A. and K. S. Kosik, Inhibition of neurite polarity by tau antisense oligonucleotides in primary cerebellar neurons. Nature, 1990. 343(6257): p. 461-3.

7. Drubin, D. G. and M. W. Kirschner, Tau protein function in living cells. J Cell Biol, 1986. 103(6 Pt 2): p. 2739-46.

8. Goedert, M. and R. Jakes, Expression of separate isoforms of human tau protein: correlation with the tau pattern in brain and effects on tubulin polymerization. Embo J, 1990. 9(13): p. 4225-30.

9. Horio, T. and H. Hotani, Visualization of the dynamic instability of individual microtubules by dark-field microscopy. Nature, 1986. 321(6070): p. 605-7.

10. Weingarten, M.D., et al., A protein factor essential for microtubule assembly. Proc Natl Acad Sci U.S.A., 1975. 72(5): p. 1858-62.

11. Kosik, K. S., Alzheimer plaques and tangles: advances on both fronts. Trends Neurosci, 1991. 14(6): p. 218-9.

12. Lee, V. M. and J. Q. Trojanowski, The disordered neuronal cytoskeleton in Alzheimer's disease. Curr Opin Neurobiol, 1992. 2(5): p. 653-6.

13. Bancher, C., et al., Accumulation of abnormally phosphorylated tau precedes the formation of neurofibrillary tangles in Alzheimer's disease. Brain Res, 1989. 477(1-2): p. 90-9.

14. Bondareff, W., et al., Molecular analysis of neurofibrillary degeneration in Alzheimer's disease. An immunohistochemical study. Am J Pathol, 1990. 137(3): p. 711-23.

15. Goedert, M., Tau protein and the neurofibrillary pathology of Alzheimer's disease. Trends Neurosci, 1993. 16(11): p. 460-5.

16. Greenberg, S. G. and P. Davies, A preparation of Alzheimer paired helical filaments that displays distinct tau proteins by polyacrylamide gel electrophoresis. Proc Natl Acad Sci U.S.A., 1990. 87(15): p. 5827-31.

17. Ksiezak-Reding, H. and S. H. Yen, Structural stability of paired helical filaments requires microtubule- binding domains of tau: a model for self-association. Neuron, 1991. 6(5): p. 717-28.

18. Lee, V. M., et al., A68: a major subunit of paired helical filaments and derivatized forms of normal Tau. Science, 1991. 251(4994): p. 675-8.

19. Kosik, K. S., Tau protein and Alzheimer's disease. Curr Opin Cell Biol, 1990. 2(1): p. 101-4.

20. Spillantini, M. G. and M. Goedert, Tau protein pathology in neurodegenerative diseases. Trends Neurosci, 1998. 21(10): p. 428-33.

21. Mailliot, C., et al., Phosphorylation of specific sets of tau isoforms reflects different neurofibrillary degeneration processes. FEBS Lett, 1998. 433(3): p. 201-4.

22. Glenner, et al., Alzheimer's Disease: Initial Report of the Purification and Characterization of a Novel Cerebrovascular Amyloid Protein, Biochem. Biophys. Res. Commun., 1984, 120:885-890.

23. Evans, D. B., et al., Tau phosphorylation at serine 396 and serine 404 by human recombinant tau protein kinase II inhibits tau's ability to promote microtubule assembly. J Biol Chem, 2000. 275(32): p. 24977-83.

### BRIEF DESCRIPTION OF THE SEQUENCE LISTING

SEQ ID NO:1 Tau 441 cDNA nucleotide sequence

SEQ ID NO:2 Tau 441 protein sequence

SEQ ID NO:3 Tau 383 cDNA nucleotide sequence

SEQ ID NO:4 Tau 383 protein sequence

SEQ ID NO:5 Tau 352 cDNA nucleotide sequence

SEQ ID NO:6 Tau 352 protein sequence

SEQ ID NO:7 Aβ 1-43 peptide sequence

### BRIEF DESCRIPTION OF THE FIGURES

FIG. 1. SPA studying the aggregation of tau in the presence of Aβ 1-42. Tau was [³³P] labeled using TPK II and then varying concentrations mixed with 2 μM Aβ 1-42 and SPA beads. The mixture was centrifuged and processed as described. CPM was plotted versus [³³P] tau concentration and the data fitted using a ligand binding 1 site equation.

FIG. 2. Time course of the aggregation of tau in the presence of Aβ 1-42. Tau (2 μM) was incubated with 60 μM Aβ 1-42 for the indicated time and the samples centrifuged. The resultant supernatant (S) and pellet (P) were run on SDS-PAGE and Coomassie stained. Lanes 1 and 2, tau alone (no Aβ 1-42) incubated for 2 hours; lanes 3 and 4, tau and Aβ 1-42 after immediate mixing; lanes 5 and 6, tau and Aβ 1-42 incubated for 15 min; lanes 7 and 8, incubated for 30 min; lanes 9 and 10, incubated for 1 hour; lanes 11 and 12, incubated for 2 hours; lane 13, MW markers.

FIG. 3. Dose-response of the aggregation of tau in the presence of varying Aβ 1-42. Tau (2 μM) or TPK II phosphorylated tau (2 μM) was incubated with varying Aβ 1-42 for 30 min at 37° C. The samples were processed as described and run on SDS-PAGE. The gel was scanned by densitometry and the percentage of tau in the pellet was calculated and plotted versus Aβ 1-42 concentration.

FIG. 4. TPK II phosphorylation of soluble tau and Aβ 1-42 aggregated tau. Tau (10 FM) was incubated in the presence of 60 μM Aβ 1-42 for 30 min at 37° C. The sample was centrifuged and the supernatant (soluble tau) removed from the pellet (aggregated tau). These two samples (2 μM each) were incubated with 3.6 nM TPK II and [³³P] incorporation followed using the filter-binding assay. Data represents the average of three experiments.

FIG. 5. Measurement of Aβ:tau interaction by absorbance measurements

FIG. 6. Phosphorylation of tau by TPK II in the presence of Aβ peptides using the filter binding assay. Tau (2 μM) was incubated with 3.6 nM TPK II and varying concentrations of either Aβ 1-42, Aβ 1-40 or Aβ 25-35 as described. The tau phosphorylation was determined by [³³P] incorporation and the percent tau phosphorylation relative to TPK II phosphorylated tau in the absence of Aβ peptides was plotted versus peptide concentration. Data represents the average of three experiments.

FIG. 7. Time course of the phosphorylation of tau by TPK II in the presence of Aβ 1-42. Tau (2 μM) was incubated with or without 60 μM Aβ 1-42 and 3.6 nM TPK II for various times. Phosphorylation was followed by [³³P] incorporation using the filter-binding assay and plotted as CPM incorporated versus time. Data represents the average of three experiments.

FIG. 8. Phosphorylation of tau by TPK II in the presence of Aβ peptides using the gel-shift assay. Tau (3 μM) was incubated with 50 nM TPK II in the presence and absence of 60 μM Aβ peptides for 4 hours and the products run on 12% SDS-PAGE as described. Lane 1, unphosphorylated tau; lane 2, TPK II phosphorylated tau; lane 3, TPK II phosphorylated tau with Aβ 25-35 ; lane 4, TPK II phosphorylated tau with non-aggregated Aβ 1-40; lane 5, TPK II phosphorylated tau with Aβ 1-42; lane 6, TPK II phosphorylated tau; lane 7, unphosphorylated tau; lane 8, MW markers.

FIG. 9. Phosphorylation of tau by TPK II in the presence of Aβ 1-42 in the gel-shift assay. Tau (3 μM) was incubated with 50 nM TPK II in the presence of varying Aβ 1-42 for 4 hours and the products run on 12% SDS-PAGE as described. Lane 1, unphosphorylated tau; lane 2, tau incubated with 60 μM Aβ 1-42; lane 3, TPK II phosphorylated tau; lanes 4-7, TPK II phosphorylated tau with 8 μm, 16 μM, 30 μM, and 60 μM Aβ 1-42 respectively; lane 8, TPK II phosphorylated tau; lane 9, unphosphorylated tau; lane 10, MW markers.

FIG. 10. Time course of the phosphorylation of tau by TPK II in the presence of Aβ 1-42 using the gel-shift assay. Tau (3 μM) was incubated with 50 nM TPK II in the presence of 60 μM Aβ 1-42 for various times and the products run on 12% SDS-PAGE as described. Lane 1, unphosphorylated tau; lane 2, TPK II phosphorylated tau for 15 min; lane 3, TPK II phosphorylated tau with Aβ 1-42 for 15 min; lane 4, TPK II phosphorylated tau for 30 min; lane 5, TPK II phosphorylated tau with Aβ 1-42 for 30 min; lane 6, TPK II phosphorylated tau for 1 hour; lane 7, TPK II phosphorylated tau with Aβ 1-42 for 1 hour; lane 8, unphosphorylated tau; lane 9, MW markers.

FIG. 11. Western blot showing that Aβ 1-42 stimulates phosphorylation of Thr-231 by TPK II. Tau (3 μM) was incubated with TPK I or TPK II in the presence and absence of 60 μM Aβ 1-42 and the samples processed as described for Western blot using the AT180 antibody. Lane 1, unphosphorylated tau; lane 2, TPK II phosphorylated tau; lane 3, TPK II phosphorylated tau with Aβ 1-42; lane 4, TPK I phosphorylated tau; lane 5, TPK I phosphorylated tau with Aβ 1-42; lane 6, TPK II and TPK I phosphorylated tau; lane 7, TPK II and TPK I phosphorylated tau with Aβ 1-42; lane 8, unphosphorylated tau.

### SUMMARY OF THE INVENTION

The invention comprises a method for identifying agents that are inhibitors of tau-beta amyloid complex formation comprising contacting a tau protein derived polypeptide and an aggregated beta-amyloid in the presence and absence of a test agent; and determining the amount of tau-beta amyloid complex formed in the presence and absence of the test agent; and comparing the amount of tau-beta amyloid complex formed in the presence of the test agent with the amount of tau-beta amyloid complex formed in the absence of the test agent.

### DETAILED DESCRIPTION OF THE INVENTION

As used hereinafter “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Glycosylated and non-glycosylated form of polypeptides are embraced by this definition.

“Synthesized” as used herein and understood in the art, refers to polynucleotides produced by purely chemical, as opposed to enzymatic, methods. “Wholly” synthesized DNA or polypeptide sequences are therefore produced entirely by chemical means, and “partially” synthesized DNAs or polypeptides embrace those wherein only portions of the resulting DNA or polypeptide were produced by chemical means.

“Isolated” as used herein and as understood in the art, whether referring to “isolated” polynucleotides or polypeptides, is taken to mean separated from the original cellular environment in which the polypeptide or nucleic acid is normally found. As used herein therefore, by way of example only, a protein expressed by recombinant means in a cell type in which it does not naturally occur is “isolated”. By way of example, a protein species, whether expressed in a naturally occurring cell or not, when purified to any extent is “isolated”

As used herein, the term “contacting” means bringing together, either directly or indirectly, a compound into physical proximity to a polypeptide or of the invention. Additionally “contacting” may mean bringing a polypeptide of the invention into physical proximity with another polypeptide

“Tau protein derived polypeptide” is a polypeptide derived from a mammalian tau comprising the microtubule binding domain of said mamamlian tau or a sequence which is homologous thereto. The microtubule binding domain is characterized by a carboxy-terminal region of three or four imperfect tandem repeats of 31 or 31 amino acids each displaying a Pro-Gly-Gly Gly motif. The term “tau protein derived polypeptide” therefore encompasses any full length mammalian tau isoform as well as fragments thereof. The structural relationship between all the human tau isoforms is elucidated by the table derived from Goedert et al. (1)

Human Tau Protein Isoforms

| | Inserts | | | |
|---|---|---|---|---|
| Isoform | 1 | 2 | 3 | Length |
| 1 | − | − | − | 352 |
| 2 | − | − | + | 383 |
| 3 | + | − | − | 381 |
| 4 | + | − | + | 412 |
| 5 | + | + | − | 410 |
| 6 | + | + | + | 441 |

The table shows the inserts contained in each isoform. SEQ ID NO:1 commprises the cDNA sequence of the longest tau isoform. Insert 1 consists of nucleotides 370-456, Insert 2 consists of nucleotides 457-543, and Insert 3 consists of nucleotides 1059-1151. SEQ ID NO:2 comprises the amino acid sequence of the longest tau isoform. Insert 1 therefore consists of amino acid 45-73, Insert 2 therefore consists of amino acid 75-102, and Insert 3 therefore consists of amino acid 275-305.

It should be clear from the foregoing that given the sequence (either DNA or protein) of the longest isoform it is possible to deduce the approximate sequence of any currently known tau isoform. By way of example, a cDNA sequence encoding tau 441 and the protein encoded is represented by SEQ ID NO:1 and 2 respectively. A cDNA sequence encoding tau 383 and the protein encoded is represented by SEQ ID NO:3 and 4 respectively. A cDNA sequence encoding tau 352 and the protein encoded is represented by SEQ ID NO:5 and 6 respectively.

The microtubule binding domain spans the area between Gln 244-Ala 390 of SEQ ID NO:2 and residues Gln 186-Ala 332 of SEQ ID NO:4 and residues Gln 186-Asn 279 of SEQ ID NO:6. The SEQ ID NOS: representing human tau isoform cDNAs and encoded proteins are included by way of example. Sequences not included are easily obtainable

For simplicity, all numbering in this patent application (unless referring to a specific SEQ ID NO.) refers to the human tau variant containing all exons (441 amino acids long) according to Goedert et al (1) and Genbank #NM_{—}005910

The invention is meant to be practiced with human and all mammalian tau isoforms (even isoforms and species variants heretofore undiscovered. Bovine tau is commercially available as a mixture of isoforms.(Sigma Cat. #T7675). A bovine tau sequence is cataloged in Genbank as accession #M26157. A mouse sequence is cataloged in Genbank as NM_{—}010838 A rat sequence is cataloged in Genbank as NM_{—}017212. Because of the high degree of homology between the tau proteins derived from other mammals it is contemplated that sequence from other species are easily obtainable via the polymerase chain reaction (PCR) via library screening and/or hybridization.

The term “beta-amyloid peptide” refers to a 39-43 amino acid peptide having a molecular weight of about 4.2 kD, which peptide is substantially homologous to the form of the protein described by Glenner, et al. (22) including mutations and post-translational modifications of the normal beta-amyloid peptide. In whatever form, the beta-amyloid peptide is an approximately 39-43 amino acid fragment (differing at the carboxy terminus of the fragment) of a large membrane-spanning glycoprotein, referred to as the beta-amyloid precursor protein (APP). Its 43-amino acid sequence is: (SEQ ID NO:7)

¹Asp Ala Glu Phe Arg His Asp Ser Gly Tyr Glu Val His His

¹⁵Gln Lys Leu Val Phe Phe Ala Glu Asp Val Gly Ser Asn Lys

²⁹Gly Ala Ile Ile Gly Leu Met Val Gly Gly Val Val Ile Ala

⁴³Thr

or a sequence which is homologous thereto. By way of example the corresponding rat and mouse sequences are about 95% homologous to the sequence above, are intended to be encompassed by this definition and can be deduced from Genbank accession number P08592 and AAB41502 respectively. Other homologues are easily deduced from public and private databases or via cloning and sequencing the corresponding APP from the appropriate species.

A beta-amyloid peptide may exist in either soluble or insoluble form. After a concentration-dependent lag period during in vitro incubations, soluble preparations of synthetic beta Aβ slowly form fibrillar aggregates that resemble natural amyloid and are separable from the aqueous medium by sedimentation. For convenience, in this specification, beta amyloid polypeptide is often referred to as Aβ 1-39, Aβ 1-40, Aβ 1-41, Aβ 1-42, Aβ 1-43 or simply “Aβ”

The term “aggregated beta-amyloid peptide” refers to beta amyloid peptides in an insoluble state.

The term “homologous” is used here to illustrate the degree of identity between the amino acid sequence of a given polypeptide and another amino acid sequence. The amino acid sequence to be compared with the amino acid sequence of the given polypeptide may be deduced from a DNA sequence, e.g. obtained by hybridization as defined above, or may be obtained by conventional amino acid sequencing methods. The degree of homology is preferably determined on the amino acid sequence of a mature polypeptide, It is preferred that the degree of homology is at least 80%, such as at least 90%, preferably at least 95% or even 98% with the amino acid sequence between the amino acid sequences compared.

Homologous amino acid sequences include those amino acid sequences which contain conservative amino acid substitutions. Percent homology can be determined by, for example, the Gap program (Wisconsin Sequence Analysis Package, Version 8 for Unix, Genetics Computer Group, University Research Park, Madison Wis.), which uses the algorithm of Smith and Waterman (Adv. Appl. Math., 1981, 2, 482-489, which is incorporated herein by reference in its entirety) using the default settings.

The term “complex” as used herein in intended to mean a combination of molecules bonded together and is not intended to convey any particular mode of bonding.

The term “tau-beta amyloid complex” or “tau-β amyloid” complex as used herein is intended to mean a complex between a tau protein derived polypeptide and a beta-amyloid peptide.

Assays of the Invention

It has long been noted that both senile plaques and neurofibrillary tangles (NFT) which are both fundamental pathological characteristic of Alzheimer's disease (AD), invariably coexist in the AD brain. The main fibrous component of all neurofibrillary lesions is paired helical filament (PHF) which contains predominantly the abnormally phosphorylated tau, while in the brain, tau is a microtubule-associated protein, which promotes microtubule assembly and stabilization. Amyloid plaques are composed of a core of β-amyloid (Aβ) peptide derived from proteolytic processing of the amyloid precursor protein, APP. There is evidence that considerable Aβ 1-42 is produced intracellularly and it is this intracellular accumulation of Aβ fibrils that may be responsible for the pathogenic process. It has been suggested that accumulation of Aβ within the cytoplasm of neurons affected by AD does not require internalization from an external source. Tau is a highly soluble cytoplasmic protein and it undergoes aggregation to form PHF-tau and NFTs. We have discovered that the addition of the aggregated form of Aβ to human recombinant soluble tau results in an instantaneous insolubility of tau protein in a dose-dependent manner. Accordingly, inhibiting the Aβ:tau interaction is a useful therapeutic intervention point in AD. This invention relates to assays and methods for studying interaction between these two molecules associated with Alzheimer's disease. It will be appreciated that assays which measure the propensity for a tau protein derived polypeptide to associate with aggregated beta-amyloid form the basis of a screening tool for agents which are effective in diminishing this interaction.

Agents that inhibit the formation of bound complexes as compared to a control binding reaction lacking agent are thereby identified as tau-Aβ aggregation inhibiting candidate therapeutic agents.

Administration of an efficacious dose of an agent capable of specifically diminishing the interaction between tau and Aβ can be used as a therapeutic or prophylactic method for treating pathological conditions (e.g.Alzheimer's Disease, Progressive Supranuclear Palsy (PSP), Corticobasal Degeneration (CBD), Down's syndrome, Frontotemporal dementia, Parkinsonism linked to chromosome 17 (FTDP), and Pick's disease ) Assays which monitor tau Aβ binding are of value in screening for therapeutic agents for the above mentioned disease states. Methods of assessing the propensity of a polypeptide like tau or derivatives of tau to associate with another polypeptide like Aβ or the formation of a tau-beta amyloid complex are well known in the art and illustrative examples are discussed.

The assays described below make use of isolated beta-amyloid either in one of it's molecular forms (typically the 39, 40, 41, 42 or 43 amino acid variants). Such polypeptides can be purchased (For example from Sigma Biochemicals—Fragment 1-38 Cat #A0189, Fragment 1-40 Cat #A1075, Fragment 1-42 Cat #A9810, Fragment 1-43 Cat #A7712) Such polypeptides may also be synthetically produced by means well known in the art. Solid state peptide synthesis is well known to those of ordinary skill in the art, and is described generally by Merrifield, 1963, J. Amer. Chem. Soc. 85:2149-2156, Fields and Noble, 1990, Int. J. Pept.. Protein Res. 35:161-214 and Solid Phase Peptide Synthesis: A practical approach” by Atherton and Sheppard (published by IRL press at Oxford University Press, 1989) which are specifically incorporated herein by reference. The peptides and proteins disclosed herein may thus be prepared using these relatively routine techniques given the disclosure of the present invention. Solid state peptide synthesis is well known to those of ordinary skill in the art, as described by the references provided and can be performed manually or by an automated peptide synthesizer such as those sold by ABS.

One method of synthesis is accomplished by solid phase peptide synthesis using the Fmoc strategy using an automated peptide synthesizer. This method involves building an amino acid chain from the —COOH terminus, which is attached to an insoluble polymeric support. The base-labile Fmoc group is used to protect the -amino group of each residue. Residues having potentially reactive side chains are protected with acid-labile groups such as t-butyl. After removal of the Fmoc group during each cycle with piperidine, the next protected amino acid is added using either a coupling reagent or pre-activated amino acid derivative. At the end of the synthesis, the peptide is cleaved from the solid support to yield a peptide acid or amide, depending on the linking agent used, and the side-chain protecting groups are removed by treating the peptide-resin with a mixture of trifluoroacetic acid and various ion scavengers. Methyl t-butyl ether is added to precipitate the peptide out of the cleavage mixture. The crude peptide is dissolved and lyophilized, after which it was purified by high performance liquid chromatography. The purified peptide is lyophilized and stored at −80° C.

Such polypeptides can also be produced as a consequence of the normal processing of the beta-amyloid precursor protein (APP) and subsequent purification.

The assays described below also make use of isolated tau protein derived polypeptides these polypeptides can be synthetically produced by means well known in the art or they may be isolated in the context of a full length tau isoform or a fragment of a tau isoform by conventional protein purification methods.

In those situations where it is preferable to partially or completely isolate the tau protein derived polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Purification of the tau protein derived polypeptide can be accomplished using a variety of techniques. If the polypeptide has been synthesized such that it contains a tag such as hexahistidine or other small peptide such as FLAG (Eastman Kodak Co., New Haven, Conn.) or myc (Invitrogen,Carlsbad, Calif.) at either its carboxyl or amino terminus, it may essentially be purified in a one-step process by passing the solution through an affinity column where the column matrix has a high affinity for the tag or for the polypeptide directly (i.e., a monoclonal antibody specifically recognizing tau). For example, polyhistidine binds with great affinity and specificity to nickel, thus an affinity column of nickel (such as the Qiagen Registered TM nickel columns) can be used for purification of tau/polyHis. (See for example, Ausubel et al., eds., Current Protocols in Molecular Biology, Section 10.11.8, John Wiley & Sons, New York [1993]).

Even if the tau protein derived polypeptide is prepared without a label or tag to facilitate purification. The tau protein derived polypeptide of the invention may be purified by immunoaffinity chromatography. To accomplish this, antibodies specific for tau must be prepared by means well known in the art. Antibodies generated against the tau protein derived polypeptide of the invention can be obtained by administering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497(1975); Kozbor et al., Immunology Today 4: 72 (1983); Cole et al., pg. 77-96 in Monoclonal Antibodies and Cancer Therapy, Alan R. Liss, Inc. (1985).

Where tau protein derived polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity.

It is understood that in some methods of detection of tau-beta amyloid complex it is desireable to label tau protein. Suitable labeling includes, but is not limited to, radiolabeling by incorporation of a radiolabeled amino acid (e.g., ¹⁴C-labeled leucine, ³H-labeled glycine, ³⁵S-labeled methionine), radiolabeling by post-translational radioiodination with ¹²⁵I or ¹³¹I (e.g., Bolton-Hunter reaction and chloramine T), labeling by post-translational phosphorylation with ³²P or ³³P (e.g., with tau protein kinase II [TPK II]) fluorescent labeling by incorporation of a fluorescent label (e.g., fluorescein or rhodamine), or labeling by other conventional methods known in the art.

One method of assessing a protein-protein interaction is a scintillation proximity assay (SPA) as described in several references, such as N. D. Cook, et al., Pharmaceutical Manufacturing International, pages 49-53 (1992); K. Takeuchi, Laboratory Practice, September issue (1992); U.S. Pat. No. 4,568,649 (1986); which are incorporated by reference herein.

### EXAMPLES

### Example 1

Measuring Tau/Aβ Interaction Using a SPA Materials

Aβ peptides and human amylin 1-37 peptide were obtained from Bachem and were dissolved and diluted in 50% DMSO GSK-3 (TPK I) was obtained from New England Biolabs. Human recombinant tau 1-383 isoform and recombinant TPK II were purified as described in Evans et al. J. Biol. Chem 275 pp. 24977-24983 (2000).

Plasmid DNA from the DE-105,2 clone (Evans et al.) was isolated and used to transform competent BL-21 (DE-3) cells used for expression with 1 mM IPTG for 4 hr in NS-85 media (23). Cells were collected by centrifugation and the cell pellets were stored at −80° C. and used for IMAC purification as described elsewhere (23). The tau protein was eluted with buffer containing 300 mM imidazole and protein containing fractions were determined by absorbance at 280 nM. Samples were run on 12% SDS-PAGE and the tau containing samples were pooled and dialyzed in 50 mM Tris, pH 8.0, 1 mM DTT.

In Vitro Reconstitution of TPK II (cdk5/p20)

In vitro reconstitution of TPK II (cdk5/p20) was performed as described in Evans et al. (23). Briefly, the full-length human cdk5 gene was inserted into the baculovirus genome using standard baculovirus expression vector technology. A sample plaque isolate producing the highest level of cdk5 was plaque purified three times and a single virus plaque was used to make stock virus for cdk5 production. For in vitro reconstitution of TPK II, human recombinant native cdk5 was partially purified by Q-Sepharose from baculovirus infected insect cells. The truncated version of human p35 activator protein, p20 (G137-R307), was cloned and expressed in E. coli as an ubiquitin fusion containing an internal hexa histidine sequence. For in vitro reconstitution and purification of human recombinant TPK II (cdk5/p20), the clone DE-93,4 containing the p20 construct, was grown in NS-85 medium (29) until the A₆₀₀=0.4 and induced for 3 h with 1 mM IPTG. The crude E. coli extract was adjusted to pH 8.0 using 2 M Tris, centrifuged at 12000×g for 1 hr and the supernatant loaded onto a nickel IMAC column equilibrated in 50 mM Tris, pH 8.0. The column was washed overnight with 50 mM Tris, pH 8.0 followed by buffer containing 50 mM imidazole and was then re-equilibrated with 50 mM Tris, pH 8.0. Partially purified cdk5 was diluted 1:2 with 50 mM Tris, pH 8.0 and loaded onto the IMAC. The column was washed with buffer containing 50 mM imidazole and the complex eluted with buffer containing 300 mM imidazole. Fractions containing TPK II (cdk5/p20) by SDS-PAGE were pooled and dialyzed in 20 mM Tris, pH 8.0, 100 mM NaCl, 1 mM DTT. Further purification of the IMAC isolated TPK II was carried out using immobilized streptavidin (Pierce) followed by affinity chromatography on an ATP agarose column (Sigma).

Phosphorylation of purified tau

Purified tau (38 μM) was incubated in the presence of 100 nM TPK II for 4 hrs at 37° C. containing 40 mM Tris, pH 7.5, 10 mM MgCI₂, 1 mM DTT, 0.5 mM ATP and 100 μCi [³³P]γATP. The unincorporated [³³P]γATP was removed by BioRad P6 spin columns using the BioRad protocol.

The Scintillation Proximity assay

[³³P] tau was added at various concentrations to 2 μM Aβ 1-42 and 5 mg/ml anti-mouse PVT SPA beads in a total volume of 100 μl of 40 mM Tris, pH 7.5, 10 mM MgCl₂, 1 mM DTT for 30 min at 37° C. The samples were centrifuged at 12,000×g in a microcentrifuge and counted using a Packard TriCarb liquid scintillation counter. The background CPM was obtained by incubating varying concentrations of [³³P] tau with the SPA beads in the absence of Aβ 1-42. Because of the insoluble nature of the tau/Aβ complex, the [³³ P] tau centrifuged into a pellet came in close proximity to the centrifuged SPA bead pellet and SPA counts were the result. Few background SPA counts were seen in the absence of Aβ 1-42 suggesting that the counts obtained were the result of an interaction of [³³P] tau and Aβ 1-42.

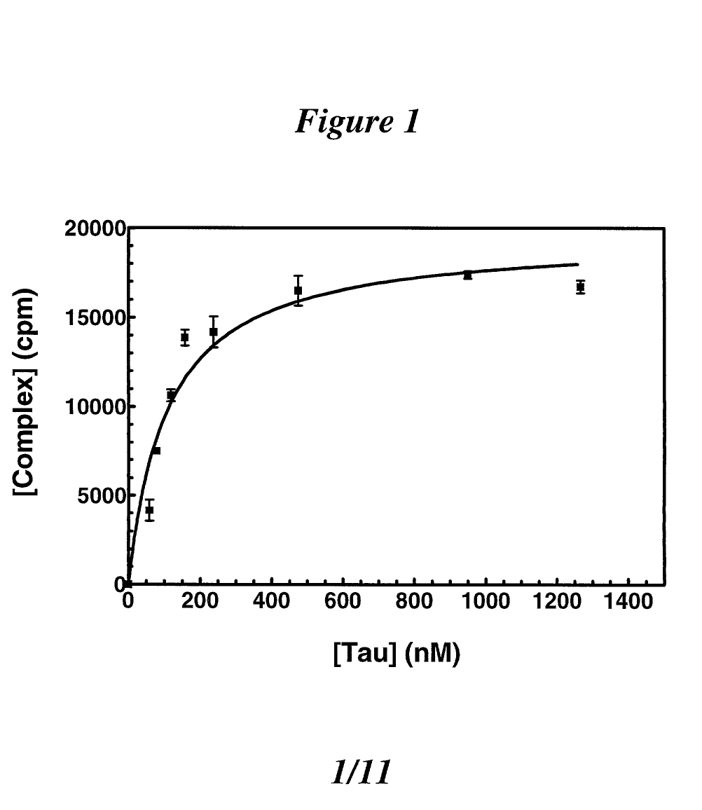

SPA counts were plotted versus [³³P] tau concentration and the data fitted using a ligand binding-1 site equation in The data obtained from the SPA assay were analyzed according to Equation (1), a rectangular hyperbola widely employed for analyzing solid phase-binding data, to determine the half-saturating level of binding (estimated dissociation constant, K_{D}) using the program Prism (GraphPad),

$$B = \frac{{B}_{max} [ L ]}{{K}_{D} + [ L ]} \qquad ( 1 )$$

where B is the complex concentration (in CPM). Bₘₐₓ is the maximum complex concentration and [L] is the molar concentration of tau. The curve is shown in FIG. 1. By varying the concentration of [³³P] tau and keeping the concentration of Aβ constant (2 μM), a relative K_{D} of 108 ±25 nM was calculated (FIG. 1). These SPA counts were inhibited in a dose dependent manner when [³³P] tau and unlabeled tau were added together to SPA beads in the presence of Aβ 1-42 (data not shown). In contrast, soluble tau was unable to compete with preformed Aβ-[³³P] tau complex, suggesting Aβ promotes tau aggregation. Our results with the above two independent techniques and using purified and well characterized tau and Aβ argue against any confounding interactions of either Aβ or tau with other assay components or proteins. These SPA counts were inhibited in a dose dependent manner when [³³P] tau and unlabeled tau were added together to SPA beads and Aβ 1-42 (data not shown). In contrast, soluble tau was unable to compete with preformed Aβ-[³³P] tau complex, suggesting the tau:Aβ interaction promotes tau aggregation.

SDS Polyacrylamide Gel Electrophoresis

The presence of a tau protein derived polypeptide/beta-amyloid peptide aggregate can be detected by analyzing the aggregate pellet and subsequent SDS-polyacrylamide gel electrophoresis or “SDS-PAGE”. SDS-PAGE is well known in the art, and is described in Laemmli, U.K., Nature 227:680 (1970), which is hereby incorporated by reference herein.

### Example 2

### Assessing Tau/Aβ Interaction by SDS-PAGE

During Aβ:tau interaction, tau undergoes a physical change from soluble tau to aggregated tau. After combining the two and centrifugation, this change can be followed by detecting tau in the pellet and supernatant fractions on a SDS-PAGE.

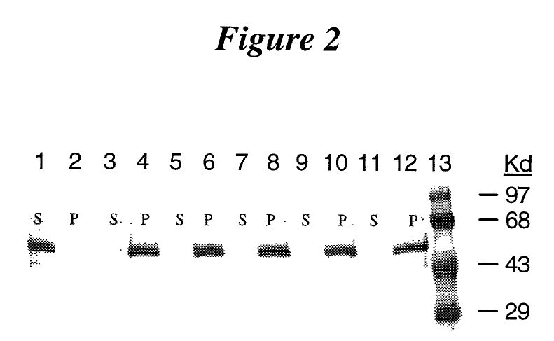

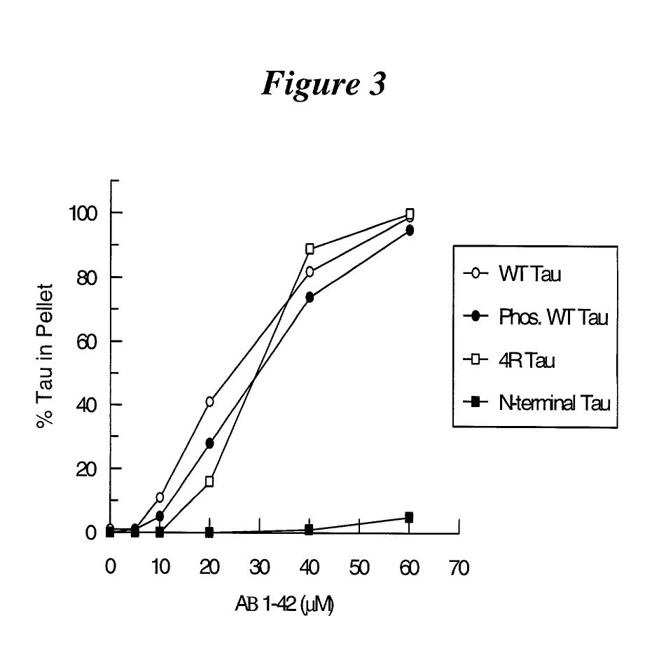

In order to perform a time course experiment human recombinant tau was incubated with aggregated Aβ 1-42 for various times under defined conditions. When Aβ:tau interaction was studied by SDS-PAGE, 2 μM tau was incubated with the indicated amount of Aβ 1-42 for the indicated time at 37° C. in a total volume of 100 μl containing 40 mM Tris, pH 7.5, 10 mM MgCl₂, 1 mM DTT. Aβ peptides were added at 10× concentrations to the assay to achieve the final concentration indicated. Samples were centrifuged at 12,000×g in a micro centrifuge for 5 min. The supernatants were removed, and the pellets washed once with 40 mM Tris, pH 7.5, 10 mM MgCI₂, 1 mM DTT and centrifuged again. The pellets were resuspended in 100 μl buffer, electrophoresed on 12% SDS-PAGE next to the corresponding supernatant, and stained with Coomassie Blue R-250. Tau bands were analyzed by densitometry. As shown in FIG. 2, Aβ 1-42-induced tau aggregation is instantaneous (lanes 3 & 4) Under these conditions, at each time point the tau protein was found to be in the pellet fraction along with Aβ 1-42. The percentage of tau protein in the pellet fraction was determined by densitometry of the SDS-PAGE. Dose response studies show that addition of Aβ 1-42 to soluble tau promotes aggregation of tau in a dose-dependent manner and can be followed by SDS-PAGE. The results of a dose-response study are shown in FIG. 3.

We recognized that [³³P] labelled tau would be convenient reagent for following the association with of tau and Aβ 1-42 . The effect of Aβ 1-42 on aggregation of unphosphorylated wild type tau was indistinguishable from TPK II phosphorylated tau (FIG. 3).

Since TPK II is a proline-directed kinase and all the proline-directed phosphorylation sites for TPK II are outside the MT-binding repeat region, we believe that Aβ 1-42 exerts its effect on tau by interacting with its MT-binding repeat domains. To test this hypothesis, four repeat (4R) tau (Q244-390A) (comprising residues 244-390 of SEQ ID NO:2 and residues 186-332 of SEQ ID NO:4) lacking the N-terminal and the C-terminal domains and N-terminal tau (M1-238S) (between residues 1-238 of SEQ ID NO:2 and 1-180 of SEQ ID NO:4 lacking the four repeats and the C-terminal domain were prepared to study the effect of Aβ 1-42 on aggregation. Aβ 1-42-induced aggregation of 4R tau (Q244-390A) was comparable to that observed for the wild type tau, while Aβ 1-42- induced aggregation was not observed with tau that lacked the MT-binding repeat domains (FIG. 3). These results suggest that MT-binding repeat domains are critical for Aβ 1-42/tau interaction, while the N-terminal tau domain (IM-238S) is not essential for Aβ 1-42-induced tau aggregation. Similar results were obtained with aggregated form of Aβ 1-40.

In the above experiments Aβ 1-42 was dissolved in DMSO. Thus, it is possible that the observed tau aggregation was due to the presence of DMSO. However, we found that the presence of DMSO had no effect on tau solubility in the absence of Aβ 1-42 under defined conditions. Likewise, the presence of DTT did not effect Aβ 1-42-induced tau aggregation. Since human tau is known to exist in at least six various isoforms and only one recombinant isoform of tau (1-383) was used in the above studies . it was of interest to determine if the observed effect of Aβ was tau isoform (1-383)-specific. Therefore, we also studied various untagged bovine tau isoforms in the presence of Aβ 1-42. The pelleted bovine tau isoforms were monitored by Western blot using 4G5 antibody to tau. These results showed that all various bovine tau isoforms were insoluble in the presence of Aβ 1-42 in a dose-dependent manner (data not shown). Since recombinant tau (1-383) used in this work contains T7 and octa His affinity tags, the results with bovine tau isoforms also confirm that the observed aggregation of human recombinant tau is indeed due to specific interaction between tau and Aβ 1-42.

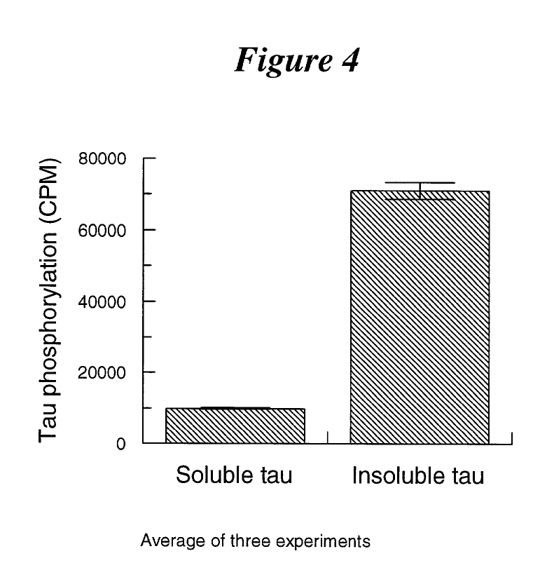

In yet another embodiment of an SDS-PAGE based assay, tau and Aβ 1-42 were mixed, centrifuged, and the tau protein in the pellet fraction was resuspended and then treated with TPK II. Using equal concentrations of tau protein substrate, FIG. 4 shows that compared to soluble tau, there was a marked increase in TPK II-mediated phosphorylation of aggregated tau due to its interaction with Aβ.

Measuring Aggregation by Turbidity Measurments

If concentrations of the tau protein derived polypeptide and the beta-amyloid peptide are sufficiently high aggregation results in changes in turbidity which can be followed over time by spectrophotometry at a suitable wavelength. The skilled artisan recognizes that the wavelength chosen is not necessarily critical because light scattering is being measured and not absorbance.

### Example 3

### Assessing Tau/Aβ Interaction by Absorbance at 350 nm

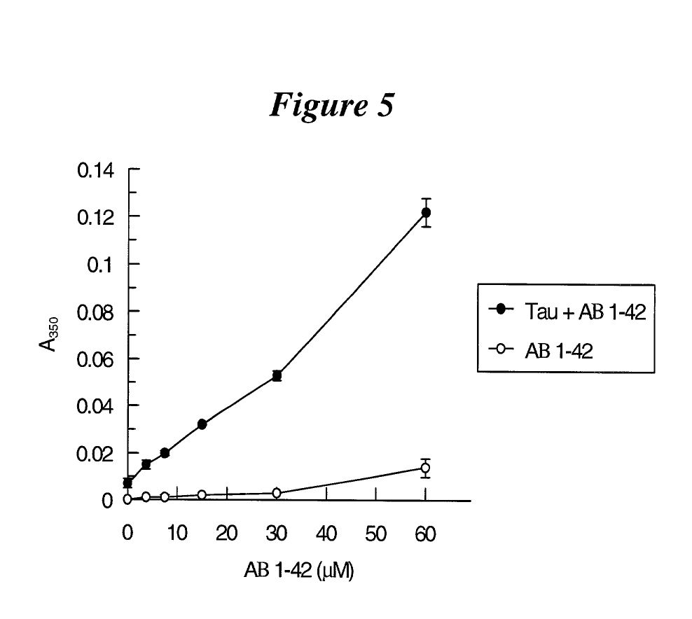

Dose-dependent Aβ 1-42-induced tau insolubility was also followed by monitoring an increase in absorbance at 350 nm as a function of Aβ 1-42 concentration. Results are shown in FIG. 5. Tau (14 μM) was mixed with varying concentrations of Aβ 1-42 (from 0 to 60 μM) in a total volume of 100 μl containing 50 mM sodium phosphate, pH 7.0. The samples were incubated in a 96 well microplate at 37° C. for 30 min and the absorbance read at 350 nm in a Molecular Devices SpectaMax Plus.

TPK II-mediated Tau Phosphorylation

The interaction between tau and Aβ can also be assessed by following an increase in phosphorylation by TPK II and is exemplified below.

### Example 4

### Assessing Tau/Aβ Interaction by following increase in phosphorylation by TPK II

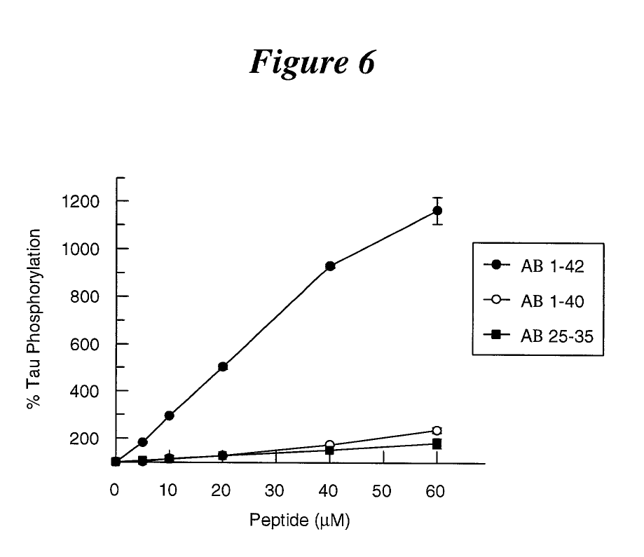

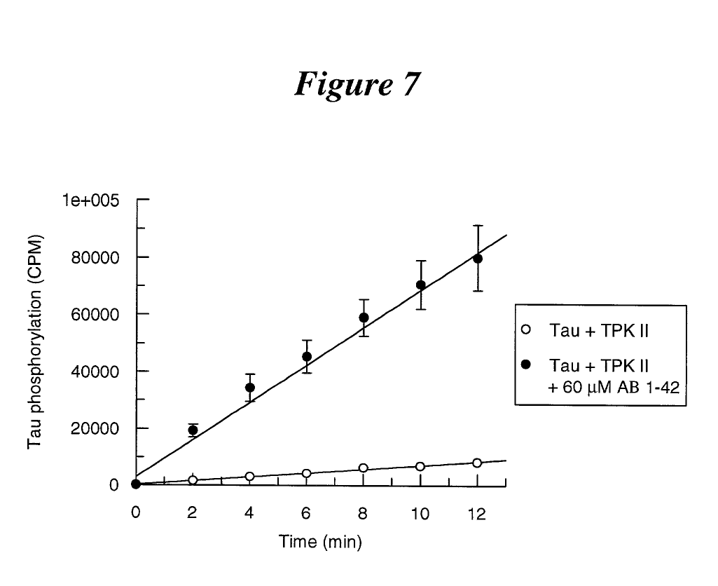

We studied the effect of Aβ 1-42 on TPK II-mediated tau phosphorylation by following incorporation of [³³P] into tau using a filter binding assay. The filter binding assay was done by incubating 2 μM tau with 3.6 nM TPK II for 10 min (or the time indicated) at 37° C. in a total volume of 15 μl containing 40 mM Tris, pH 7.5, 10 mM MgCl₂, 1 mM DTT, 0.1 mg/ml BSA and 1 μCi [³³P]γATP. Aβ peptides were added at 10× concentrations to the assay to achieve the final concentration indicated. After incubation, 10 μl was removed and spotted onto phosphocellulose paper. The papers were washed 3× with 1% phosphoric acid, once with acetone, dried and counted in a Packard Tricarb liquid scintillation counter. FIG. 6 shows that under defined conditions, Aβ 1-42 stimulates TPK II-mediated tau phosphorylation by about 8 to 10-fold. There was only about a 2-3 fold stimulation of tau phosphorylation by non-aggregated form of Aβ 1-40 or Aβ 25-35 . FIG. 7 shows a time course study demonstrating that in the presence of Aβ 1-42 there is about an 8-fold increase in the initial rate of tau phosphorylation by TPK II.

Gel-Shift Assays

Aggregation and phosphorylation can be accessed by a gel shift assay (an assay in which complex formation is accessed by a decrease in electrophoretic migation of complexed vs. uncomplexed Aβ. An illustrative example is shown below.

### Example 5

### Assessing Tau/Aβ Interaction by Gel-Shift

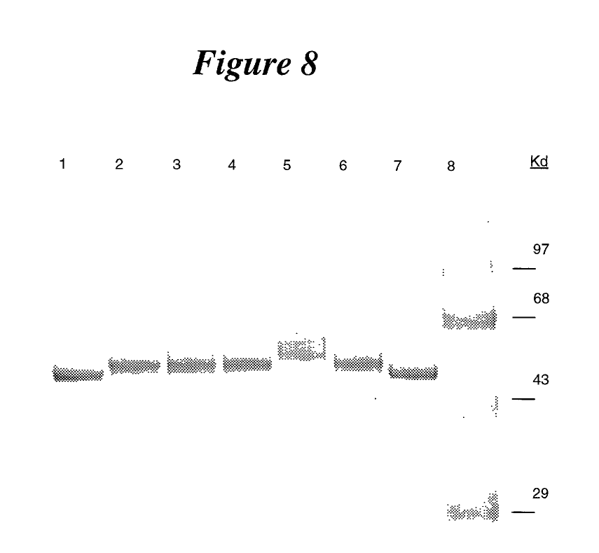

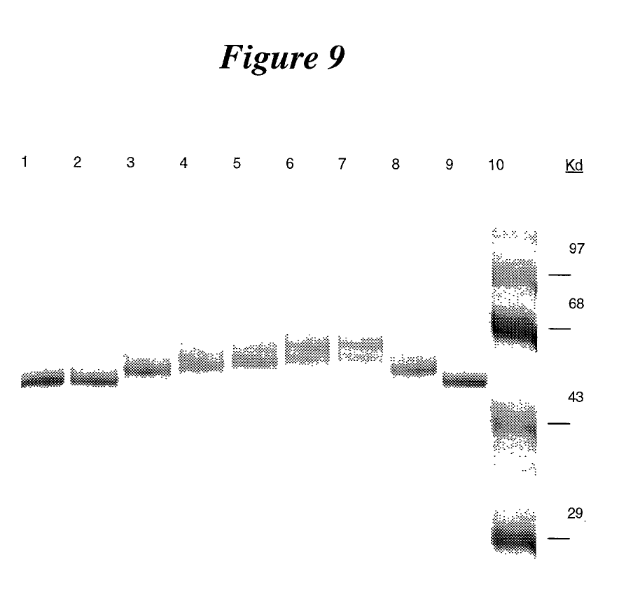

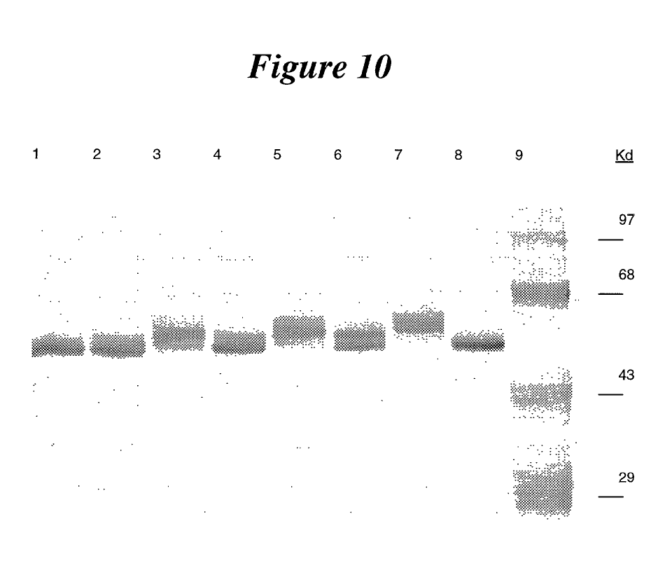

In another embodiment, increase in TPK II phosphorylation was followed by a gel-shift assay. Gel-shift assays were done by incubating 3 μM tau with 50 nM TPK II and/or 167 units/ml TPK I for the indicated time at 37° C. in a total volume of 20 μl containing 40 mM Tris, pH 7.5, 10 mM MgCl₂, 1 mM DTT, and 1 mM ATP. Aβ peptides were added at 10× concentrations to the assay to achieve the final concentration indicated. Following incubation the samples were electrophoresed on 12% SDS-PAGE and stained with Coomassie Blue R-250. As shown in FIG. 8, a significant increase in gel shift was observed in the presence of Aβ 1-42 (lane 5). The control peptides, non-aggregated Aβ 1-40 or 25-35 peptide, had no effect on tau in the gel shift assay. These results were confirmed with aggregated Aβ 1-42 purchased from two other commercial sources. The observed increase in tau phosphorylation in the presence of Aβ 1-42 was shown to be both dose (FIG. 9) and time-dependent (FIG. 10), consistent with that shown above in the filter binding assays and SDS-PAGE assays.

Techniques Utilizing Antibodies to Tau Derived Polypeptides

Antibodies to native tau or a phosphorylated epitope of phosphorylated tau as can be helpful in determining the propensity of a tau derived polypeptide to associate with Aβ The interaction can the be determined by western blot or by ELISA. The techniques for performing Western blots are well known in the art and to those of skill in the art. The detection of the interaction in a sample may also be carried out by using some of the well known ELISA principles, e.g. direct, catching, competitive and double enzyme linked immunosorbent assay.

### Example 6

### Assessing Tau/Aβ Interaction By Western Blot

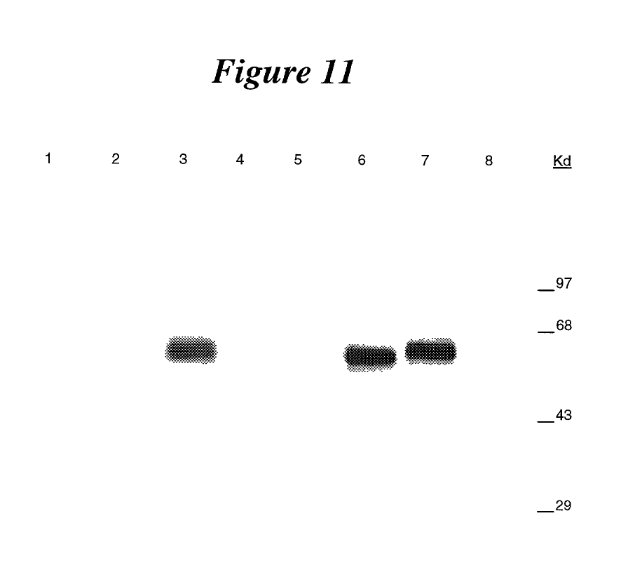

In yet another embodiment, Western blots can be used to follow Aβ :tau interaction by monitoring phosphorylation of an AD-related phosphoepitope (Thr-231) on human recombinant tau. The Western blots were done by using the same phosphorylation conditions as the gel-shift assay. After incubation for 4 hours, approximately 300 ng tau was electrophoresed on 12% SDS-PAGE, transferred to nitrocellulose and blocked with 4% BSA. AT180 antibody was used at a 1:5000 dilution followed by goat anti mouse IgG-peroxidase used at a 1:10000 dilution as the secondary antibody. The blots were developed using the ECL protocol. Phosphorylation of an AD-related epitope (P-Thr-231) in tau can be detected by the AT-180 monoclonal antibody, which almost exclusively recognizes paired helical filaments (PHF's) from AD brain (Goedert 1994). FIG. 11 shows that Aβ 1-42 stimulates phosphorylation of Thr-231 by TPK II. Furthermore, studies with a T231 A tau mutant confirmed that stimulation of phosphorylation at Thr-231 was indeed due to an interaction between Aβ and wild type tau.

### Example 7

### Tau/Aβ Interaction by ELISA

A number of ELISA formats can be used to study tau:Ap interaction. In one embodiment, Aβ is coated onto microtiter plates at 60 μg/ml dissolved in 50% DMSO in 50 mM sodium carbonate buffer, pH 9.6. After washing three times with 0.05% Tween 20, a 2% solution of milk extract in PBS (137 mM sodium chloride/10 mM psohphate/2.68 mM KCl, pH 7.4) was added as a blocker. The plate is washed three times with PBS and varying concentrations of tau (1 to 10 μM) in PBS are added. After 1 hr of incubation at 37 degree C., the plate is washed with PBS. The tau:Aβ interaction can followed by using antibodies to full length tau and a commercally available secondary antibody and a standard ELISA kit. In another embodiment, phosphorylated tau can be used and antibodies to phoshorylated tau can be used as a source of primary antibody. In yet another embodiment. A tagged version of tau can be used and antibodies to the tag can be used as a primary antibody. In a further embodiment, tau can be bound to the solid phase as described above for Aβ. In this case, Aβ is captured by tau on the plates and antibodies to Aβ can be used as a source of primary antibody. In another embodiment tau is captured on the plates and antibodies to tau can be used as a primary antibody.

Fluorescence Polarization

When a fluorescently labeled molecule is excited with plane polarized light, it emits light that has a degree of polarization that is inversely proportional to its molecular rotation. Large fluorescently labeled molecules remain relatively stationary during the excited state (4 nanoseconds in the case of fluorescein) and the polarization of the light remains relatively constant between excitation and emission. Small fluorescently labeled molecules rotate rapidly during the excited state and the polarization changes significantly between excitation and emission. Therefore, small molecules have low polarization values and large molecules have high polarization values. It is to be expected then that as a fluorescently labelled molecule is incorporated into an aggregate its polarization value would increase.

### Example 8

The interaction of tau with Aβ 1-42 was studied using fluorescence polarization. Tau was fluorescently labeled using the Alexa 488 protein labeling kit from Molecular Probes. Alexa labeled tau (16 nM) was incubated with varying concentrations of Aβ 1-42 (from 0 to 50 μM) for 30 min at 37° C. in 40 mM Tris, pH 7.5, 10 mM MgCl₂, 1 mM DTT. The reactions were read on a fluorescence polarization instrument from Panvera and an increase in polarization studied as a function of increasing Aβ 1-42 concentration. In another embodiment Aβ 1-42 can be synthesized to contain a fluorescein label and the increase in polarization studied as a function of increasing tau concentration.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention. It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

What is claimed is:

1. A method for identifying agents that are inhibitors of tau-beta amyloid complex formation comprising:
(a) contacting a tau protein derived polypeptide and an aggregated beta-amyloid peptide in the presence and absence of a test agent; and
(b) determining the amount of tau-beta amyloid complex formed in the presence and absence of the test agent; and
(c) comparing the amount of tau-beta amyloid complex formed in the presence of the test agent with the amount of tau-beta amyloid complex formed in the absence of the test agent wherein a test agent which decreases the amount of tau-beta amyloid complex formed is an inhibitor.

2. The method of claim 1 wherein said tau derived polypeptide comprises residues 244-390 of SEQ ID NO:2

3. The method of claim 1 wherein said tau derived polypeptide comprises residues 186-332 of SEQ ID NO:4

4. The method of claim 1 wherein said tau derived polypeptide comprises residues 186-279 of SEQ ID NO:6

5. The method of claim 1 wherein said aggregated beta-amyloid peptide comprises residues 1-39 of SEQ ID NO:7

6. The method of claim 1 wherein said aggregated beta-amyloid peptide comprises residues 1-40 of SEQ ID NO:7

7. The method of claim 1 wherein said aggregated beta-amyloid peptide comprises residues 1-41 of SEQ ID NO:7

8. The method of claim 1 wherein said aggregated beta-amyloid peptide comprises residues 1-42 of SEQ ID NO:7

9. The method of claim 1 wherein said aggregated beta-amyloid peptide comprises residues 1-43 of SEQ I) NO:7

10. The method of claim 1 wherein the amount of tau-beta amyloid complex is determined by scintillation proximity assay.

11. The method of claim 1 wherein the amount of tau-beta amyloid complex is determined by polyacrylamide gel electrophoresis.

12. The method of claim 1 wherein the amount of tau-beta amyloid complex is determined by turbidity measurement

13. The method of claim 1 wherein the amount of tau-beta amyloid complex is determined by gel shift assay.

14. The method of claim 1 wherein the amount of tau-beta amyloid complex is determined by antibody binding.

15. The method of claim 1 wherein the amount of tau-beta amyloid complex is determined by ELISA.

16. The method of claim 1 wherein the amount of tau-beta amyloid complex is determined by Western blotting

17. The method of claim 1 wherein the amount of tau-beta amyloid complex is determined by fluorescence polarization.

18. The method of claim 1 wherein the amount of tau-beta amyloid complex is determined by fluorescence polarization

19. The method of claim 1 wherein the tau protein derived polypeptide is labeled.

20. The method of claim 1 wherein the tau protein derived polypeptide comprises a tag.


SEQUENCE LISTING (7 sequences)

SEQ ID NO: 1 - DNA, 2796 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 2 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - DNA, 1200 bp, Homo sapiens
tgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggt

SEQ ID NO: 4 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - DNA, 2529 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 6 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 43 aa, Homo sapiens
DAEFRHDSGYEVHHQKLVFFAEDVGSNKGAIIGLMVGGVVIAT